(19) United States
(12) Patent Application Publication
(10) Pub. No.: US 2023/0076407 A1
(43) Pub. Date: Mar. 9, 2023

(54) PHYSICAL PERFORMANCE ASSESSMENT METHOD AND DEVICE THROUGH MOTION ACCELERATION SENSOR ATTACHED TO HEAD

(71) Applicant: BEFLEX INC., Daejeon (KR)

(72) Inventor: Changkeun JUNG, Daejeon (KR)

(21) Appl. No.: 17/759,797

(22) PCT Filed: Dec. 22, 2020
(86) PCT No.: PCT/KR2020/018900

§ 371 Date: Jul. 29, 2022

(30) Foreign Application Priority Data
Jan. 31, 2020 (KR) 10-2020-0011661

(51) Int. Cl. A61B 5/11; G01P 15/18; A61B 5/00

(52) U.S. Cl. CPC A61B 5/112; G01P 15/18; A61B 5/4023; A61B 5/1116; A61B 5/6814; A61B 5/7275; A61B 2562/0219

(57) ABSTRACT

The present invention relates to a physical performance assessment method including the steps of: collecting a patient's motion acceleration signals for the patient's motion analysis measured from a motion acceleration sensor attached to the patient's head through a motion measurement module; and deriving a plurality of motion parameters based on the collected motion acceleration signals through a motion analysis module.

### TECHNICAL FIELD

The present invention relates to a physical performance assessment method and device through a motion acceleration sensor attached to the head, and more specifically, to a physical performance assessment method and device through a motion acceleration sensor attached to the head that is capable of assessing a patient's physical performance through the motion acceleration sensor attached to the patient's head, thereby improving the degree of accuracy in analyzing his or her physical performance.

### BACKGROUND ART

As aging societies have been developed fast all over the world, older people who have medical/surgical treatments due to their aging have been drastically increased. Aging changes the structures of the cells and tissues of all of organs of the body and decreases their functions. For example, aging has a relation with sarcopenia and low physical strength, which makes a quality of life deteriorated.

To overcome such aging problems, many kinds of medical devices, treatment agents, and programs for improving the physical performance of older patients have been studied and developed.

So as to appropriately assess the physical performance of an older patient, it is important to accurately determine a degree of the patient's frailty.

So as to determine a degree of frailty, in conventional practices, short physical performance battery (SPPB) is used to measure the patient's gait speed, chair stand, and balance and thus determine his or her physical performance.

However, professionally trained personnel (hereinafter, referred to as medical staff) is needed in executing comprehensive geriatric assessment for older patients, and further, a lot of time is disadvantageously required for the assessment. Besides, the patient's physical performance is measured with the naked eye of the medical staff, and accordingly, low accuracy in the assessment may be made. Further, the assessment results may be varied according to the patient's mood or condition upon the test, and the frailty of the patient is assessed through indirect data, thereby making the degree of accuracy in the assessment results diminished.

Therefore, there is a need to develop a physical performance assessment method for calculating test data with accuracy, irrespective of surrounding situations.

### DISCLOSURE

### Technical Problem

Accordingly, the present invention has been made in view of the above-mentioned problems occurring in the related art, and it is an object of the present invention to provide a physical performance assessment method and device through a motion acceleration sensor attached to a patient's head that is capable of effectively improving the degree of accuracy in measuring the patient's motions.

### Technical Solution

To accomplish the above-mentioned objects, according to one aspect of the present invention, there is provided a physical performance assessment method using a physical performance assessment device having a motion measurement module for measuring a patient's motion acceleration signals through a motion acceleration sensor attached to the patient's head and a motion analysis module for analyzing the patient's physical performance based on the measured motion acceleration signals, the method including the steps of: collecting the motion acceleration signals measured from the motion acceleration sensor attached to the patient's head through the motion measurement module and used to analyze the patient's motions; and deriving a plurality of motion parameters based on the collected motion acceleration signals through the motion analysis module, wherein the step of deriving the plurality of motion parameters comprises the steps of: if a protocol for balance among the plurality of motion parameters is executed, collecting the patient's three-axis motion acceleration signals for side-by-side stance, tandem stance, and semi-tandem stance and detecting balance parameters capable of assessing the patient's balance performance from total acceleration signals where the three-axis motion acceleration signals are added; if a protocol for gait speed among the plurality of motion parameters is executed, detecting single stance and double stance from the motion acceleration signals to detect gait parameters from the detected results; and if a protocol for chair stand among the plurality of motion parameters is executed, detecting a minimum peak value and a maximum peak value from the up and down acceleration and the total motion acceleration signals to detect stand parameters through the relation between the minimum peak value and the maximum peak value.

According to the present invention, the physical performance assessment method may further include the step of determining, if the protocol for the balance is executed, whether a balance test is completed based on the magnitude of the total acceleration signals with respect to a preset threshold value.

According to the present invention, if the magnitude of the total acceleration signals is greater than the preset threshold value, it is determined that the test fails or the patient quits in the middle of the test, the time taken is recorded up to the time point where the magnitude of the total acceleration signals is greater than the threshold value, and scores are applied to the recorded time according to preset score ranges.

According to the present invention, the magnitude of the total acceleration signals is an amplitude difference between a maximum peak value and a minimum peak value in the total acceleration signals.

According to the present invention, the physical performance assessment method may further include the steps of: detecting the patient's gait time points based on the gait parameters; sensing a gait start point and a gait finish point based on the gait time points; and assessing the patient's gait speed based on the time taken from the gait start point and the gait finish point.

According to the present invention, while the protocol for the chair stand is executed, if the minimum peak value is sensed earlier than the maximum peak value in the up and down acceleration and the total motion acceleration signals so that a difference between the minimum peak value and the maximum peak value is greater than the preset threshold value, it is determined that a stand-to-sit motion is performed.

According to the present invention, while the protocol for the chair stand is executed, if the maximum peak value is sensed earlier than the minimum peak value in the up and down acceleration and the total motion acceleration signals so that a difference between the minimum peak value and the maximum peak value is greater than the preset threshold value, it is determined that a sit-to-stand motion is performed.

According to the present invention, the stand parameters comprise stand-to-sit parameters for the stand-to-sit motion and sit-to-stand parameters for the sit-to-stand motion.

According to the present invention, the gait parameters further comprise vertical ground reaction forces and peak values and trough values of the vertical ground reaction forces.

According to the present invention, the gait parameters further comprise low frequency power in low frequency band and high frequency power in high frequency band, and according to a rate of the low frequency power with respect to the high frequency power, the patient's gait motion is determined.

According to the present invention, if the constant value is less than a preset constant value capable of determining the patient's gait motion, it is determined as a normal state, and if the constant value is greater than the preset constant value, it is determined that the patient's physical performance stability becomes deteriorated.

To accomplish the above-mentioned objects, according to another aspect of the present invention, there is provided a physical performance assessment device including: a motion measurement module for collecting a patient's motion acceleration signals for the patient's motion analysis through a motion acceleration sensor attached to the patient's head; and a motion analysis module for deriving a plurality of motion parameters based on the collected motion acceleration signals, wherein the motion analysis module collects, if a protocol for balance among the plurality of motion parameters is executed, the patient's three-axis motion acceleration signals for side-by-side stance, tandem stance, and semi-tandem stance to detect balance parameters capable of assessing the patient's balance performance from total acceleration signals where the three-axis motion acceleration signals are added, detects, if a protocol for gait speed among the plurality of motion parameters is executed, single stance and double stance from the motion acceleration signals to detect gait parameters from the detected results, and detects, if a protocol for chair stand among the plurality of motion parameters is executed, a minimum peak value and a maximum peak value from the up and down acceleration and the total motion acceleration signals to detect stand parameters through the relation between the minimum peak value and the maximum peak value.

Objects, characteristics and advantages of the present invention will be more clearly understood from the detailed description as will be described below and the attached drawings.

### Advantageous Effects

According to the present invention, the physical performance assessment method and device can divide the patient's motions based on the patient's motion acceleration signals collected from the motion acceleration sensor attached to the patient's head and assess the patient's physical performance, thereby minimizing an error rate.

Additionally, the physical performance assessment method and device is configured to distinguish the gait time points for the left and right legs from each other, based on the motion acceleration collected from the motion acceleration sensor attached to the patient's head, thereby increasing the conveniences of the assessment.

Further, the physical performance assessment method and device is configured to collect the up and down acceleration, the front and back acceleration, and/or the left and right acceleration so that the patient's motions can be completely analyzed to accurately detect his or her gait abnormality due to a disease.

The effectiveness of the invention is not limited as mentioned above, and it should be understood to those skilled in the art that the effectiveness of the invention may include another effectiveness as not mentioned above from the detailed description of the present invention.

### BRIEF DESCRIPTION OF DRAWINGS

FIGS. 1a and 1b are schematic views showing a physical performance assessment device through a motion acceleration sensor attached to the head according to the present invention.

FIG. 2 is a block diagram showing the physical performance assessment device according to the present invention.

FIG. 3 is a flowchart showing a physical performance assessment method through a motion acceleration sensor attached to the head according to the present invention.

FIG. 4 is a schematic diagram showing a balance test protocol according to the present invention.

FIG. 5a is an exemplary view showing balance parameters derived by axis according to the present invention.

FIG. 5b is an exemplary view showing the test parameters based on the balance test according to the present invention, wherein the test fails.

FIG. 5c is an exemplary view showing the test parameters based on the balance test according to the present invention, wherein the test is completed.

FIG. 6 is a schematic diagram showing a gait speed test protocol according to the present invention.

FIG. 7 is a graph showing motion acceleration by time according to the present invention.

FIG. 8 is an exemplary view showing a process of collecting gait acceleration signals to detect gait time points according to the present invention.

FIG. 9 is a flowchart showing a gait parameter detection method according to the present invention.

FIG. 10 is a graph showing the power by frequency for three axes according to the present invention.

FIG. 11 is a schematic diagram showing a chair stand protocol according to the present invention.

FIG. 12 is a graph showing motion acceleration by time according to the present invention.

FIG. 13 is an exemplary view showing a stand-to-sit motion according to the present invention, which corresponds to an area A of FIG. 12.

FIG. 14 is an exemplary view showing a sit-to-stand motion according to the present invention, which corresponds to an area B of FIG. 12.

### BEST MODE FOR INVENTION

Embodiments of the present invention just provide the principle of the present invention. Therefore, the implementation of the principle of the present invention and various devices included in the concept and scope of the present invention may be carried out by a person having ordinary skill in the art. Further, it should be appreciated that all terms and embodiments described in the present invention are obviously intended to allow the concept of the present invention to be understood and are not limited by the disclosed embodiments of the present invention.

In the description, terms, such as the first, the second, and the like may be used to describe various elements that are in the same situation, independently of each other, and it should understood that no main/sub or master/slave positions are given to the elements.

Objects, characteristics and advantages of the present invention will be more clearly understood from the detailed description as will be described below and the attached drawings, and therefore, the technical spirit of the present invention can be easily implemented by a person having ordinary skill in the art.

Some or all of the features of various embodiments of the present invention may be combined to one another, and they may technically operate cooperatively with one another, which will be understood by a person having ordinary skill in the art. Further, the respective embodiments of the present invention may be carried out independently of each other or combinedly with each other.

Hereinafter, exemplary embodiments of the present invention will be described in detail below with reference to the accompanying drawings.

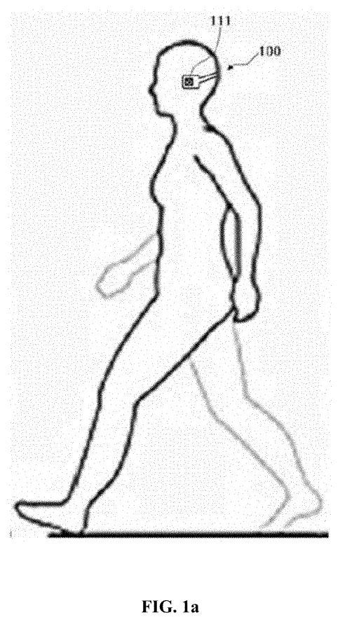

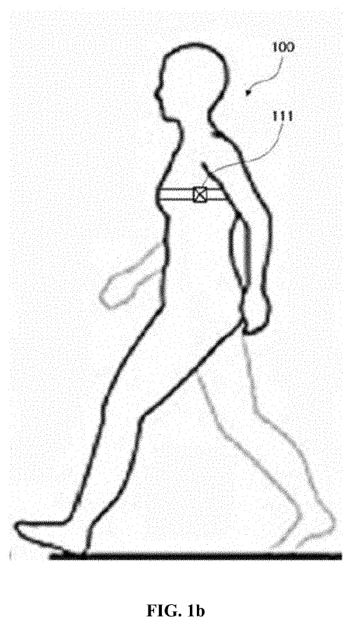

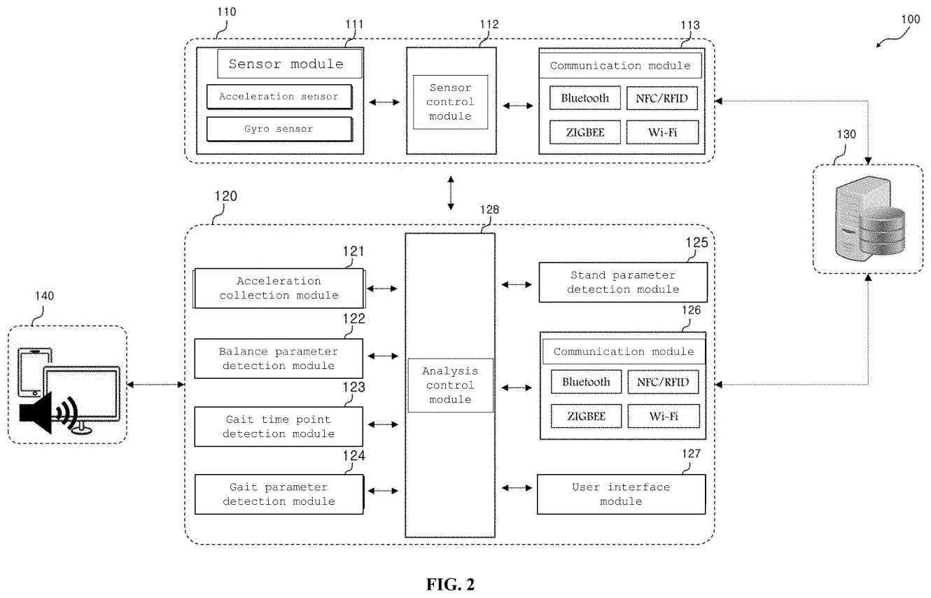

Now, an explanation of a configuration of a physical performance assessment device 100 according to the present invention will be given with reference to FIGS. 1a to 2.

### Explanation of Physical Performance Assessment Device

FIGS. 1a and 1b are schematic views showing a physical performance assessment device through a motion acceleration sensor attached to the head according to the present invention. FIG. 2 is a block diagram showing the physical performance assessment device according to the present invention.

Referring to FIGS. 1a and 1b, the physical performance assessment device 100 according to the present invention serves to assess the physical performance of older patients based on objective assessment metrics such as balance, gait speed, and chair stand. That is, the physical performance assessment device 100 is defined as a device for executing a short physical performance battery (SPPB) protocol. In this case, the SPPB protocol is an assessment tool (or a measurement protocol) that combine the results of three objective assessment metrics, that is, balance, gait speed, and chair stand, which are usefully and easily measured among the physical performance assessment methods for older patients. According to the present invention, the SPPB protocol can be called ‘physical performance assessment tool’, for the conveniences of the description. An explanation of the physical performance assessment method using the physical performance assessment tool will be given later.

The physical performance assessment device 100 may be a wearable device having a motion acceleration sensor attached to a headset, an earphone, a cap, or the like, which is worn on a patient's head. As shown in FIG. 1a, the physical performance assessment device 100 is attached to the headset. However, of course, the physical performance assessment device 100 may be worn on the patient's waist or upper body around the chest (See FIG. 1b), without being limited thereto.

Referring to FIG. 2, the physical performance assessment device 100 includes a motion measurement module 110, a motion analysis module 120, a database 130, and a notification module 140.

The motion measurement module 110 serves to measure the patient's motion acceleration signals using the motion acceleration sensor attached to his or her head. In this case, the motion acceleration signals may be up and down motion acceleration az, left and right motion acceleration ax, and front and back motion acceleration ay.

Referring to FIG. 1a, the motion measurement module 110 is attached to the headset. However, the motion measurement module 110 may be detachably attached to the patient's upper body around the chest through a band (See FIG. 1b). Accordingly, the motion measurement module 110 may be attached to the upper body as well as the head. For example, the motion measurement module 110 may be attached to the patient's upper arm, ear, head, or shoulder through a band or the like. However, if the motion measurement module 110 is detachably attached to the body region under the upper body, the degree of accuracy in measurement may be diminished, and accordingly, the motion measurement module 110 is desirably attached to the body region close to the head, excepting the lower body. According to the present invention, further, the motion measurement module 110 may be accommodated in a separate housing from the motion analysis module 120 or in the same housing as the motion analysis module 120.

Referring to FIG. 2, the motion measurement module 110 includes a sensor module 111, a sensor control module 112, and a communication module 113.

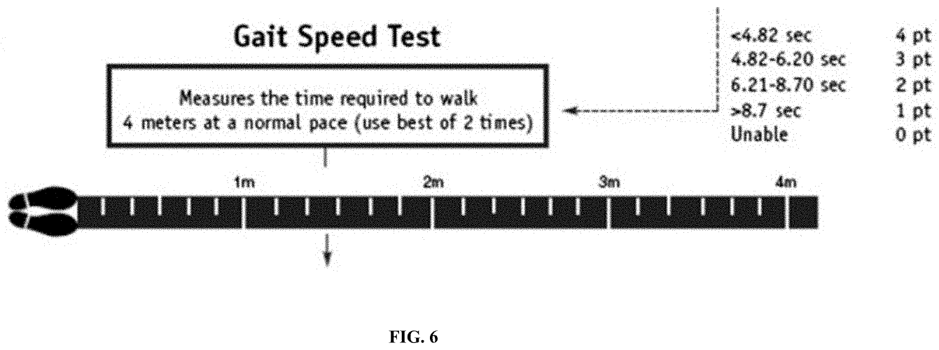

The sensor module 111 serves to measure the sensing values (hereinafter, referred to as acceleration signals or sensor values) required for the motion analysis of the motion analysis module 120 and transmit the measured values to the motion analysis module 120. According to the present invention, sensing the sensor values required for gait analysis through the sensor module 111 will be described, but the sensor module 111 collects the sensed motion acceleration, detects gait time points and gait parameters from the collected motion acceleration, and transmits the detected results to the motion analysis module 120. Further, the sensor module 111 includes a motion acceleration sensor and a gyro sensor, and of course, the sensor module 111 may measure the sensing values through a GPS sensor. A process of detecting the gait time points and the gait parameters will be explained later with reference to FIG. 6.

The sensor control module 112 serves to control all operations of the motion measurement module 110.

The communication module 113 serves to transmit the sensor values sensed by the sensor module 111 to the motion analysis module 120. For example, the communication module 113 includes Bluetooth, Near Field Communication (NFC), Radio-Frequency Identification (RFID), Zigbee module, Wi-Fi, and the like.

Referring to FIG. 2, the motion analysis module 120 serves to analyze physical performance analysis information based on the motion measurement module 110. The physical performance analysis information is defined as information of the patient's gait speed, balance, and stand-to-sit and sit-to-stand performance analyzed, based on the SPPB protocol, and it should be desirably understood that the physical performance analysis information is different from motion parameters as will be discussed later.

In specific, the motion analysis module 120 includes a motion acceleration collection module 121 for receiving the motion acceleration signals from the sensor module 111 of the motion measurement module 110 to collect motion acceleration, a balance parameter detection module 122 for deriving balance parameters from the collected motion acceleration, a gait time point detection module 123 for detecting minimum and maximum peaks of the motion acceleration from the collected motion acceleration to detect gait time points, a gait parameter detection module 124 for deriving gait parameters based on the detected gait time points, and a stand parameter detection module 125 for sensing stand-to-sit and sit-to-stand motions from the collected motion acceleration to detect stand-to-sit parameters and sit-to-stand parameters.

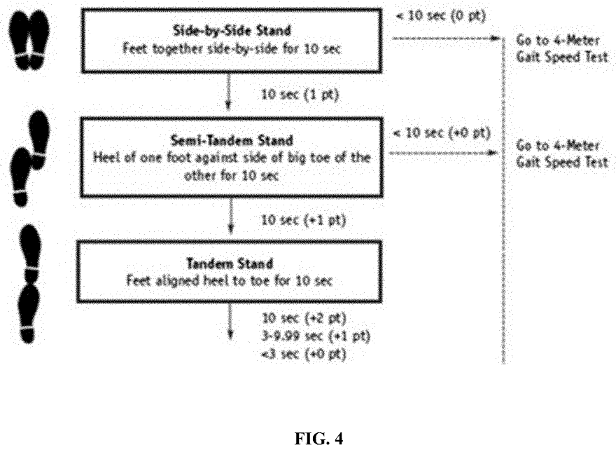

Moreover, the motion analysis module 120 may further include a communication module 126 for transmitting signals to the motion measurement module 110 and the notification module 140 of the physical performance assessment device 100. The specific operations of the gait time point detection module 123 and the gait parameter detection module 124 will be explained later with reference to FIGS. 4 to 14.

If the motion acceleration collection module 121 is provided integrally with the motion measurement module 110, the communication module 113 of the motion measurement module 110 is connected directly to the motion analysis module 120 to transmit the collected motion acceleration signals to the motion analysis module 120. Further, the gait analysis information is outputted through the notification module 140. For example, the gait analysis information may be outputted through a speaker, a smartphone, a computer, wireless earphones, and the like.

The notification module 140 serves to convert test guiding information for the SPPB protocol for assessing the patient's physical performance through the detection of various parameters using the motion analysis module 120 into the form recognized by the patient, such as sound or video and output the converted information. For example, if a balance test is performed, the notification module 140 produces a sound, “Please stand side-by-side on a floor” through the speaker provided in the physical performance assessment device 100 or the smartphone, computer, or wireless earphones connected to the physical performance assessment device 100, or displays the notification information through a display module.

Further, the notification module 140 serves to convert the physical performance analysis information produced from the motion analysis module 120 into the information recognized by the patient, such as sound or video, and output the converted information. For example, if there is a need to correct a step length, a sound, “Please reduce a step length” is outputted through the speaker or the smartphone, computer, or wireless earphones, and otherwise, an alarm sound such as a beep sound may be outputted. In this case, if a step length to be corrected is long, the alarm sound is outputted at a fast speed, and contrarily, if a step length to be corrected is short, the alarm sound is outputted at a slow speed. Through the alarm sound, accordingly, the patient can recognize the step length to be corrected and correct his or her step length. In specific, the notification module 140 includes the wireless earphones or the speaker, but according to the present invention, basically, the motion acceleration sensor is attached to the earphones worn on the patient's head, so that the physical performance analysis information can be transmitted directly to the patient's ear through the physical performance assessment device 100. Further, the notification module 140 is connected to the smartphone, computer, dedicated display, or the like to output the correction information to the form of the video, and accordingly, the correction information may be outputted to various forms.

Further, the physical performance assessment device 100 transmits the physical performance analysis information derived by the motion analysis module 120 to the database 130. In this case, the physical performance analysis information is accumulatedly stored so that the gait positions of the patient can be checked according to time evolution.

If large pieces of physical performance analysis information are accumulatedly stored, the stored data is utilized as big data that can be used for all kinds of statistical analysis. The physical performance assessment device 100 has an analysis control module 128 for controlling all operations of the motion analysis module 120.

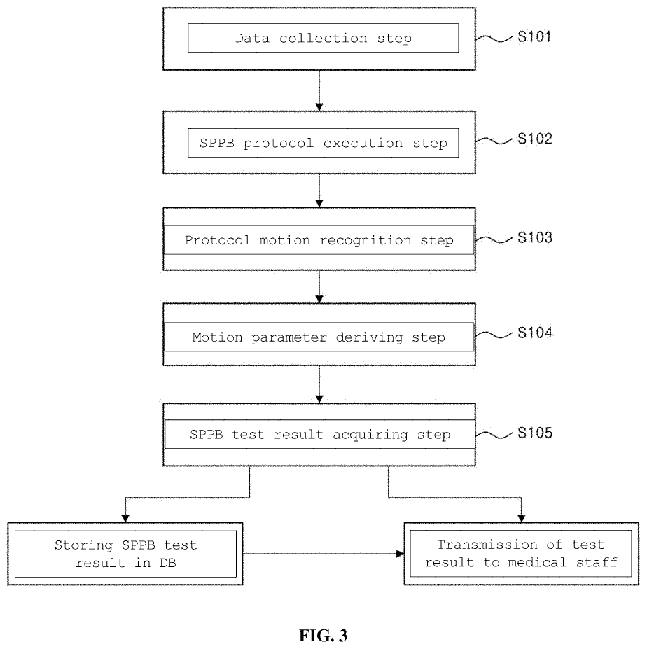

Hereinafter, an explanation of a physical performance assessment method using the SPPB protocol according to the present invention will be given with reference to FIG. 3.

FIG. 3 is a flowchart showing a physical performance assessment method according to the present invention.

Referring to FIG. 3, the motion measurement module 110 collects data from the physical performance assessment device 100 attached to the patient's head (at step S101).

Next, the motion analysis module 120 executes the SPPB protocol (at step S102). Through the SPPB protocol, the three assessment metrics as mentioned above, that is, balance, gait speed, and chair stand are executed, and an explanation of the SPPB protocol will be given in detail later with reference to FIGS. 4 to 14.

Next, the motion analysis module 120 recognizes the patient's motions by protocol (at step S103) and derives motion parameters (at step S104). In this case, the motion parameters include balance parameters, gait parameters, and stand parameters as a plurality of metrics derived from the SPPB protocol. In this case, each parameter includes at least one metric.

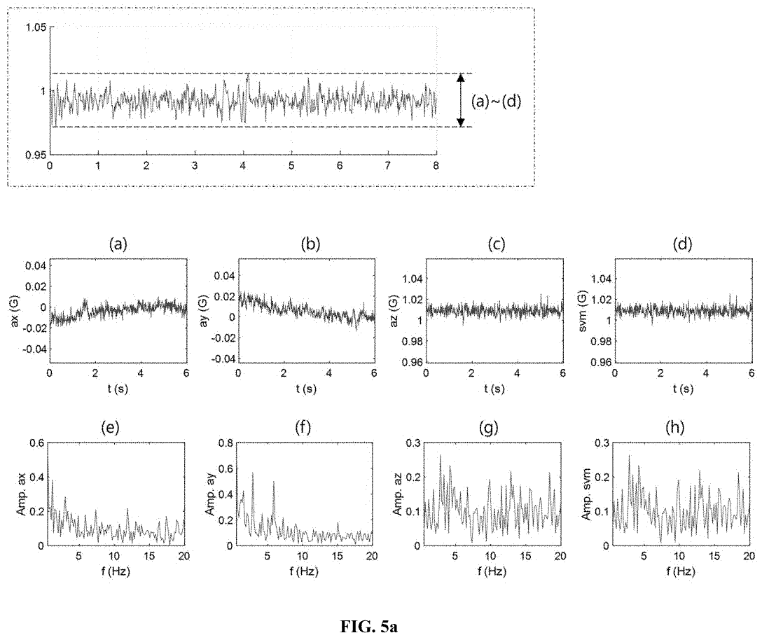

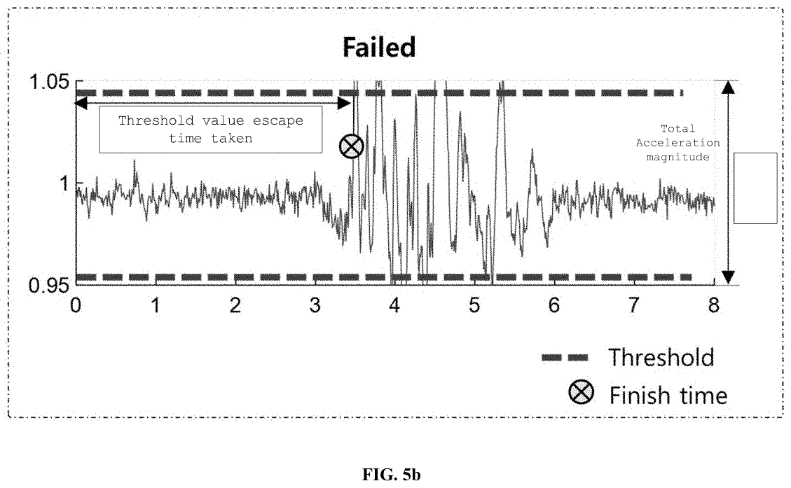

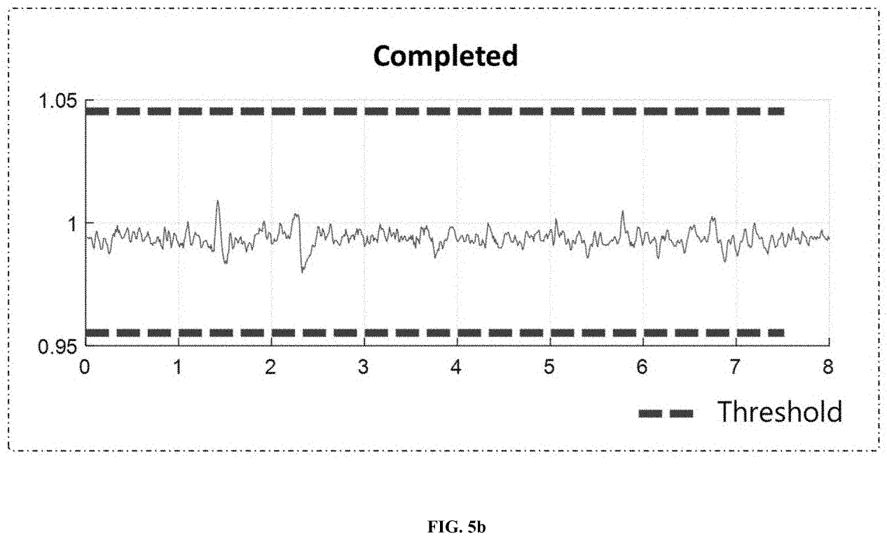

If the balance test is carried out, the protocol motions for three positions of the patient, that is, side-by-side stance, tandem stance, and semi-tandem stance are recognized, and based on the recognized positions, the balance parameters can be derived. In this case, the side-by-side stance is a position where the upper limbs come into contact with the trunk in an upright standing position with the legs open to a shoulder width, the semi-tandem stance is a position where one foot is placed slightly further ahead the other foot, and the tandem stance is a position where one foot is placed directly in front of the other foot, with the toes of one foot almost touching the heel of the other foot. An explanation of the balance test will be given in detail later with reference to FIGS. 4 to 5c.

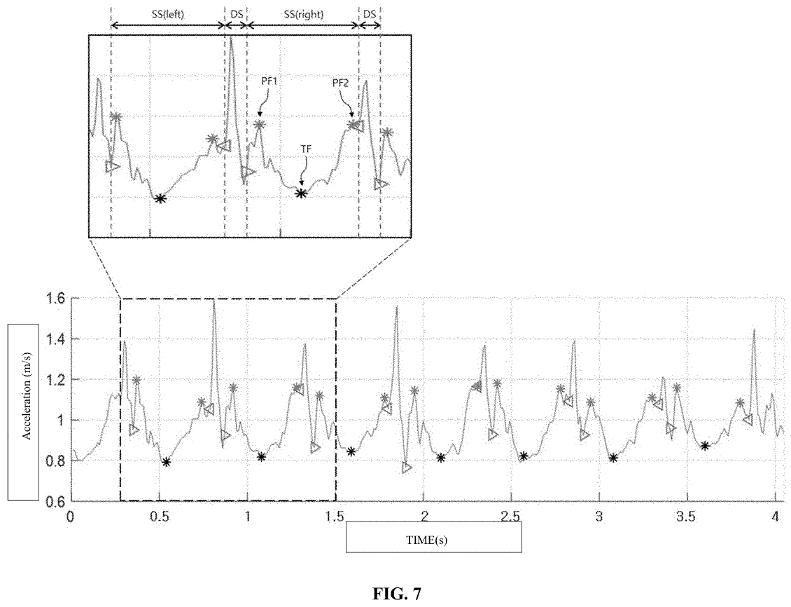

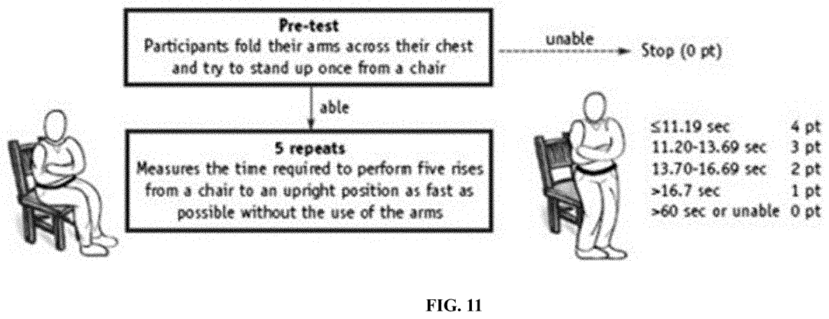

If the gait test is carried out, the patient's gait time points are detected based on his or her motion acceleration to recognize the gait motion including the gait peed of the patient, and based on the recognized motion, the gait parameters can be derived. The gait parameters include detection components for analyzing the gait motion of the patient based on the detected gait time points, for example, double support (DS), single support (SS), cadence, and step width. An explanation of the patient's gait parameters will be given in detail later with reference to FIG. 7. If stand-to-sit and sit-to-stand tests are carried out, further, stand-to-sit and sit-to-stand motions are recognized based on the patient's up and down acceleration or the analysis value of the signal vector magnitude, and based on the recognized motions, a plurality of stand parameters can be derived. An explanation of the stand-to-sit and sit-to-stand tests will be given in detail later with reference to FIGS. 11 to 14.

Next, the SPPB test results are acquired (at step S105), and the obtained results are stored in the database DB or transferred to the medical staff. In this case, after the acquired results are stored in the database DB, they may be transferred to the medical staff through a wired or wireless network.

Now, an explanation of a process of executing the SPPB protocol for the balance test according to the present invention will be given in detail with reference to FIGS. 4 to 5c.

SPPB Protocol for Balance Test

FIG. 4 is a schematic diagram showing a balance test protocol according to the present invention. FIG. 5a is an exemplary view showing the balance parameters derived by axis according to the present invention. FIG. 5b is an exemplary view showing the test parameters based on the balance test according to the present invention, wherein the test fails. FIG. 5c is an exemplary view showing the test parameters based on the balance test according to the present invention, wherein the test is completed. FIGS. 5b and 5c show total acceleration signals (hereinafter referred to as “motion acceleration signals”) that are obtained by adding the acceleration signals by axis.

The balance test is carried out by repeatedly executing the SPPB protocol for the three positions. That is, the motion parameters for the side-by-side stance, the semi-tandem stance, and the tandem stance are derived, whether each position is held for 10 seconds is assessed, and scores are applied to the assessed results, which are repeatedly executed.

In specific, the test is started through the patient's input. In this case, the patient's input is performed by the smartphone or PC connected to the physical performance assessment device 100 or by a button additionally included in the physical performance assessment device 100. In this case, an explanation of the test protocol will be given to the patient. For example, the explanation of the test protocol is outputted to the form of sound through the speaker module included in the physical performance assessment device 100 or to the form of text, image, video, and the like through the smartphone or PC connected to the physical performance assessment device 100.

Further, as shown in FIG. 4, the assessment is carried out in the order of the side-by-side stance, the semi-tandem stance, and the tandem stance, but if necessary, it may be changed in order. When the position is changed, the patient goes to 4-meter so that the gait speed test can be carried out together. An explanation of the gait speed test will be given later.

Referring to FIG. 5a, the patient's motion acceleration signals on the respective axes ax, ay, and az are collected in the order of assessment as mentioned above. For example, the left and right acceleration signals ax (See the graph (a) of FIG. 5a), the up and down acceleration signals az (See the graph (b) of FIG. 5a), and the front and back acceleration signals ay (See the graph (c) of FIG. 5a) are collected. In this case, the balance parameters are detected from the motion acceleration signals through the balance parameter detection module 122, and the detectable balance parameters are listed in Table 1.

TABLE 1

| | Parameter | Description |
|---|---|---|
| (a) | ax amplitude | Difference between max and |
| | | min of ax signals |
| (b) | ay amplitude | Difference between max and |
| | | min of ay signals |
| (c) | az amplitude | Difference between max and |
| | | min of az signals |
| (d) | svm amplitude | Difference between max and |
| | | min of svm signals |
| (e) | ax high | Power sum of signals having |
| | frequency power | 0.5 Hz or more |
| (f) | ay high | Power sum of signals having |
| | frequency power | 0.5 Hz or more |
| (g) | az high | Power sum of signals having |
| | frequency power | 0.5 Hz or more |
| (h) | svm high | Power sum of signals having |
| | frequency power | 0.5 Hz or more |

Referring to a graph (d) of FIG. 5a, further, if the collected three axis acceleration signals ax, ay and az are added, the total motion acceleration magnitude (Signal Vector Magnitude (SVM)) is calculated. For example, it can be appreciated that the total motion acceleration magnitude is about 0.4. In this case, the total motion acceleration magnitude is calculated by the following mathematical expression 1.

|v|=\sqrt{x²+y²+z²} <Mathematical expression 1>

In this case, as shown in graphs (e) to (h) of FIG. 5a, the frequencies of the signals by axis are analyzed to thus analyze shaking in the positions. Generally, in the standing position on both feet, if shaking of 0.1 Hz or less occurs, it means that stable position is held, and if shaking is in the range of 0.1 to 0.5 Hz, it means that the vestibular organ is abnormal. Further, if shaking is in the range of 0.5 to 1 Hz, it means that somatic senses from the lower limb and spine are abnormal, and if shaking of 1 Hz or more occurs, the central nervous system is abnormal. So as to measure the balance abnormality of older patients, accordingly, the acceleration signals by axis for the frequencies f(Hz) of 0.5 Hz or more and the total acceleration magnitude as the sum of the acceleration signals are collected, as shown in the graphs (e) to (h) of FIG. 5a. As a result, the total motion acceleration magnitude can be recognized.

After that, as shown in FIGS. 5b and 5c, it is determined whether the test is completed or fails based on the acceleration magnitude with respect to preset threshold values. In this case, the preset threshold values represent the amplitude of the acceleration signals preset by the patient, and in FIGS. 5b and 5c, the preset threshold values are about 0.1. However, of course, the preset threshold values may be changed according to embodiments of the present invention.

In specific, referring to FIG. 5b, if an amplitude difference between a maximum peak and a minimum peak of the total acceleration signals is greater than the preset threshold values, it is determined that the test fails or the patient quits in the middle of the test, and accordingly, the time taken is recorded up to the time point where the amplitude difference is over the threshold values to provide scores. In this case, if the amplitude difference is less than the threshold values, it is determined that the test is completed, and accordingly, the test is finished. That is, if the amplitude difference is less than the threshold values, it is desirably determined that the patient's motions are stable.

Further, if scores are applied to the total acceleration signals collected according to the three positions, they are applied according to preset score ranges. According to the preset score ranges, if the side-by-side and semi-tandem positions are held for 10 seconds or more, one point (1 pt) is applied to each position, and if the tandem position is held for three seconds or more, one point (1 pt) is applied. Further, if the tandem position is held for 10 seconds or more, two points (2 pt) are applied, and the maximum score is set to four points. In this case, desirably, the preset score ranges are generally based on the SPPB used to determine the frailty of the older patient.

Accordingly, the motion analysis module 120 extracts the shaking in the three axis positions through the balance parameters detected from the balance parameter detection module 122, and the scores are applied according to the patient's position holding degrees for given time, thereby assessing the balance performance of the patient.

Now, an explanation of the gait speed test according to the present invention will be given in detail with reference to FIGS. 6 to 10.

SPPB Protocol for Gait Speed Test

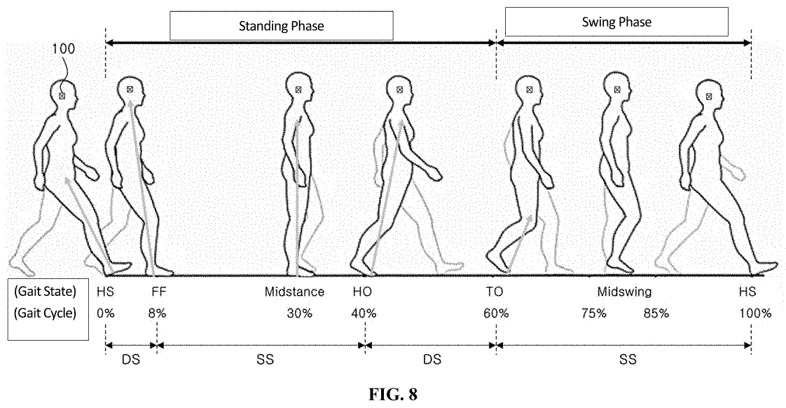

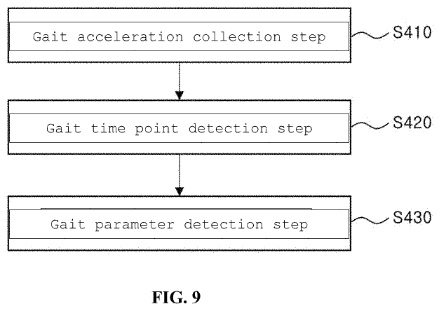

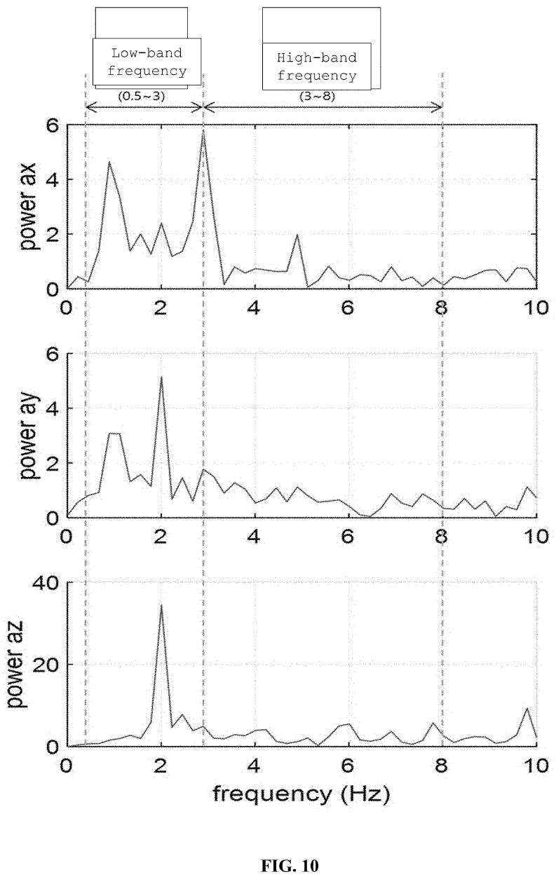

FIG. 6 is a schematic diagram showing a gait speed test protocol according to the present invention. FIG. 7 is a graph showing motion acceleration by time according to the present invention. FIG. 8 is an exemplary view showing a process of collecting gait acceleration signals to detect gait time points according to the present invention. FIG. 9 is a flowchart showing a gait parameter detection method according to the present invention. FIG. 10 is a graph showing the power by frequency for three axes according to the present invention. Referring first to FIG. 7, a horizontal axis represents time s, and a vertical shaft acceleration m/s². When the gait speed test is carried out, the process of notifying test start and test explanation is the same as in the above-mentioned balance test, and therefore, an explanation of the process will be avoided.

Referring to FIG. 6, the gait speed test is carried out by measuring the time (sec) taken to walk 4 meters in a standing position at a normal pace. Accordingly, the patient's gait start and finish points are sensed to record the time taken, and based on the time taken, scores are applied. For example, if the test fails, a zero point is applied, and if the time taken is greater than 8.7 seconds, one point is applied. If the time taken is in the range of 6.21 to 8.7 seconds, two points are applied, if the time taken is in the range of 4.82 to 6.20 seconds, three points, and if the time taken is less than 4.82 seconds, four points.

For example, if it is assumed that there is a patient walks 4 meters within four seconds, the gait acceleration signals of the patient are collected (at step S910 of FIG. 9), the gait time points are detected from the collected gait acceleration signals through the gait time point detection module 123 (at step S920), and the gait parameters are detected through the gait parameter detection module 122 (at step S930). In this case, the gait parameters related to the detectable gait time points are listed in Table 2.

TABLE 2

| | Parameter | Description |
|---|---|---|
| (a) | Single stance time | Time during which single |
| | | stance is maintained |
| (b) | Single stance time | Time during which single |
| | left | stance (left foot) is |
| | | maintained |
| (c) | Single stance time | Time during which single |
| | right | stance (right foot) is |
| | | maintained |
| (d) | Double stance time | Time during which double |
| | | stance is maintained |
| (e) | Double stance time | Time during which double |
| | left | stance is maintained (after |
| | | single left foot stance) |
| (f) | Double stance time | Time during which double |
| | right | stance is maintained (after |
| | | single right foot stance) |
| (g) | Single stance time | Variance for time during which |
| | variance | single stance is maintained |
| (h) | Single stance time | Variance for time during which |
| | left variance | single stance (left foot) is |
| | | maintained |
| (i) | Single stance time | Variance for time during which |
| | right variance | single stance (right foot) is |
| | | maintained |
| (j) | Double stance time | Variance for time during which |
| | variance | double stance is maintained |
| (k) | Double stance time | Variance for time during which |
| | left variance | double stance is maintained |
| | | (after single left foot |
| | | stance) |
| (l) | Double stance time | Variance for time during which |
| | right variance | double stance is maintained |
| | | (after single right foot |
| | | stance) |

In specific, referring to FIG. 7, the patient's motion acceleration signals from a landing time point [symbol] to the intermediate time point [symbol] of a ground reaction force peak are defined as single stance SS. In this case, the single stance includes the single stance on the left foot SS(left) and the single stance on the right foot SS(right), and the respective single stances are periodically repeated. Further, the rest stance excepting the single stance is defined as double stance DS.

Like this, the total acceleration signals including the single stance SS and the double stance DS are defined as a gait cycle. As shown in FIG. 8, the gait cycle is divided into a standing phase and a swing phase, and during one gait cycle, the single stance SS and the double stance DS appear two times, respectively. Further, if the gait speed of the patient becomes faster, the single stance SS becomes increased, and the double stance SS becomes decreased. Also, the standing phase includes five stages such as heel strike (HS), foot flat (FF), midstance, heel off (HO), and toe off (TO). Further, the swing phase includes three stages such as toe off (TO), midswing, and heel strike (HS). According to the present invention, the gait stages are described with respect to the right foot, and the left foot is described as the opposite side to the right foot.

Further, the gait parameter detection module 124 detects a ground reaction force peak value and a ground reaction force trough value as the gait parameters. Referring to FIG. 7, there are three peaks * in the single stance SS, that is, between the minimum point [symbol] and the maximum point [symbol] of the acceleration. In this case, a light gray peak * located on one single stance SS (or located on the left side of one single stance SS) represents a first vertical ground reaction force peak value PF1, a dark gray peak * located on one single stance SS (or located on the right side of one single stance SS) represents a second vertical ground reaction force peak value PF2, and a black peak * located between the peaks located on the left and right sides of one single stance SS) represents a vertical ground reaction force trough value TF1. The gait parameters are listed in Table 3.

TABLE 3

| | Parameter | Description |
|---|---|---|
| (a) | First peak force | Peak value of first vertical |
| | | ground reaction force |
| (b) | First peak force | Peak value of first vertical |
| | left | ground reaction force (left foot) |
| (c) | First peak force | Peak value of first vertical |
| | right | ground reaction force (right foot) |
| (d) | Trough force | Trough value of vertical ground |
| | | reaction force |
| (e) | Trough force left | Trough value of vertical ground |
| | | reaction force (left foot) |
| (f) | Trough force right | Trough value of vertical ground |
| | | reaction force (right foot) |
| (g) | Second peak force | Peak value of second vertical |
| | | ground reaction force |
| (h) | Second peak force | Peak value of second vertical |
| | left | ground reaction force (left foot) |
| (i) | Second peak force | Peak value of second vertical |
| | right | ground reaction force (right foot) |
| (j) | First peak force | Variance of peak value of first |
| | variance | vertical ground reaction force |
| (k) | First peak force | Variance of peak value of first |
| | left variance | vertical ground reaction force |
| | | (left foot) |
| (l) | First peak force | Variance of peak value of first |
| | right variance | vertical ground reaction force |
| | | (right foot) |
| (m) | Trough force | Variance of trough value of |
| | variance | vertical ground reaction force |
| (n) | Trough force left | Variance of trough value of |
| | variance | vertical ground reaction force |
| | | (left foot) |
| (o) | Trough force right | Variance of trough value of |
| | variance | vertical ground reaction force |
| | | (right foot) |
| (p) | Second peak force | Variance of peak value of second |
| | variance | vertical ground reaction force |
| (q) | Second peak force | Variance of peak value of second |
| | left variance | vertical ground reaction force |
| | | (left foot) |
| (r) | Second peak force | Variance of peak value of second |
| | right variance | vertical ground reaction force |
| | | (right foot) |

Further, the gait parameter detection module 124 detects the power by frequency band with respect to the three-axis acceleration ax, ay, and az as the gait parameters. The gait parameters are listed in Table 4.

TABLE 4

| | Parameter | Description |
|---|---|---|
| (a) | ax low frequency | Low frequency power |
| | power | (0.5 to 3 Hz) |
| (b) | ax high frequency | High frequency power |
| | power | (3 to 8 Hz) |
| (c) | ay low frequency | Low frequency power |
| | power | (0.5 to 3 Hz) |
| (d) | ay high frequency | High frequency power |
| | power | (3 to 8 Hz) |
| (e) | az low frequency | Low frequency power |
| | power | (0.5 to 3 Hz) |
| (f) | az high frequency | High frequency power |
| | power | (3 to 8 Hz) |

In specific, as shown in FIG. 10, it can be appreciated that the power at low frequency band (in the range of 0.5 to 3 Hz) is higher than that at high frequency band (in the range of 3 to 8 Hz) according to the power by frequency band (power ax, power ay, and power az) with respect to the three-axis acceleration (ax, ay, and az). In this case, the motions at the low frequency band are defined as the frequencies which have a high probability that the motions are intended by a person, and the motions at the high frequency band are defined as the frequencies caused by the motions not intended by a person, for example, shaking.

In this case, a rate of the low frequency band power with respect to the high frequency band power is calculated by the following Mathematical expression 2 and obtained as a constant value. In this case, if the constant value is less than a preset constant value, it is determined as a normal state, and if greater than the preset constant value, it is determined as an abnormal state.

$$\frac{\begin{matrix}High - band frequeqncy \\ ( square of total power in the 3 - 8 Hz band )\end{matrix}}{\begin{matrix}Low - band frequency \\ ( square of total power in the 0.5 - 3 Hz band )\end{matrix}} = Constant \qquad < Mathematical expression 2 >$$

In this case, the preset constant value is 0.5, and if the constant value obtained through the Mathematical expression 2 is less than 0.5, the normal state is determined. However, if greater than 0.5, it is possible to have neurological diseases (for example, Parkinson's disease).

Referring to the power by frequency band (power ax) with respect to the left and right motion acceleration of FIG. 10, the power at low frequency band (in the range of 0.5 to 3 Hz) is present in the range of about 1 to 6, and the power at high frequency band (in the range of 3 to 8 Hz) is present in the range of about 0 to 2. As mentioned above, that is, the rate of the low frequency band power with respect to the high frequency band power is less than 0.5, it can be determined as a normal state.

Further, the gait parameter detection module 124 further detects the gait parameters as listed in Table 5. In this case, the gait parameters (a) to (f) of Table 5 are disclosed in detail in Korean Patent No. 10-2055661, which are suggested for the reference of the present invention. The vertical oscillations listed in the gait parameters (h) to (j) of Table 5 can be calculated by Mathematical expression 3.

TABLE 5

| | Parameter | Description |
|---|---|---|
| (a) | Step count | The number of steps |
| (b) | Cadence | The number of steps per |
| | | minute |
| (c) | Speed | Speed |
| (d) | Step length | Step length |
| (e) | Step width | Step width |
| (f) | Head angle | Vertical inclination |
| | | angle of head |
| (g) | Head angle | Variance of vertical |
| | variance | inclination angle of |
| | | head |
| (h) | Vertical | Vertical |
| | oscillation | oscillation |
| (i) | Vertical | Vertical oscillation |
| | oscillation | (left) |
| | left | |
| (j) | Vertical | Vertical oscillation |
| | oscillation | (right) |
| | right | |

Vertical oscillation=Constant×Central frequency power oscillation <Mathematical expression 3>

Accordingly, the motion analysis module 120 detects the gait time points from the gait parameters detected through the gait parameter detection module 124, and based on the gait time points, the start and finish points are sensed. As a result, scores are applied according to the time taken from the start point to the finish time of the gait time points, thereby assessing the patient's gait performance.

SPPB Protocol for Chair Stand Test

Hereinafter, an explanation of the chair stand test according to the present invention will be given with reference to FIGS. 11 to 14. The chair stand test includes tests for a stand-to-sit motion and a sit-to-stand motion.

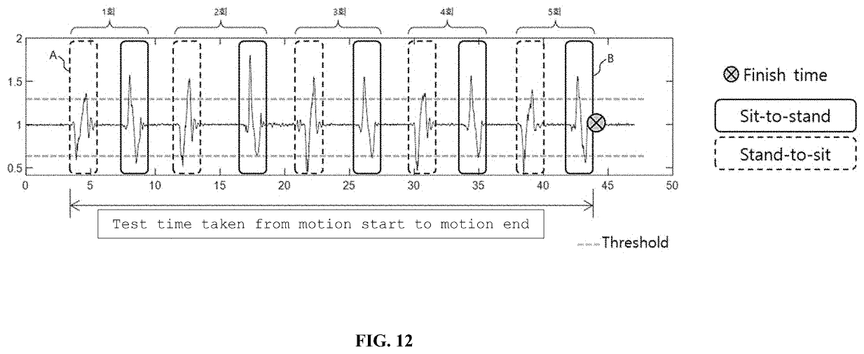

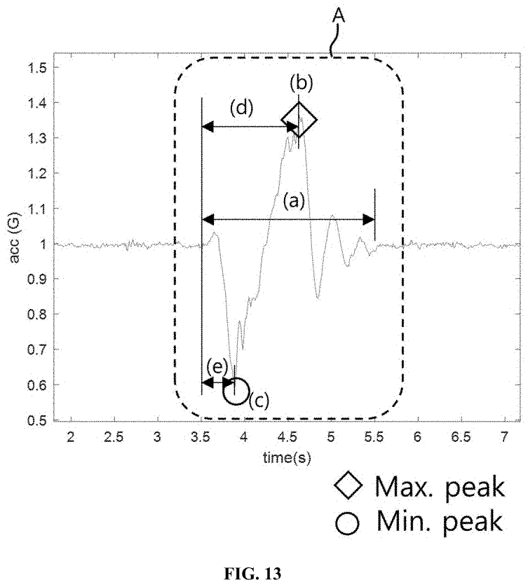

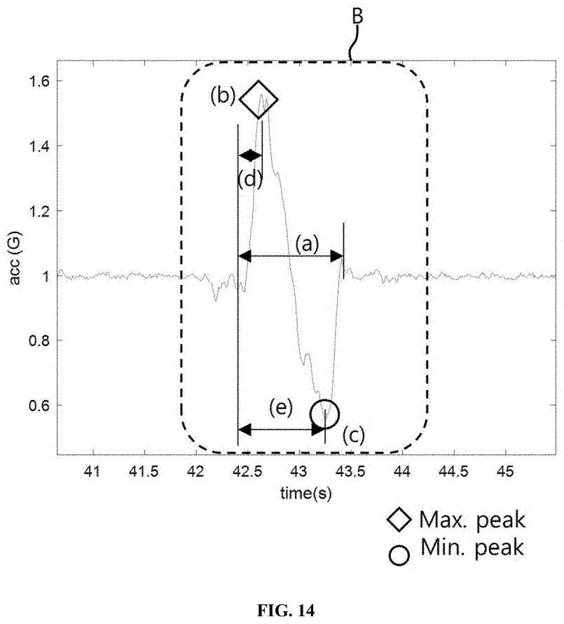

FIG. 11 is a schematic diagram showing a chair stand protocol according to the present invention. FIG. 12 is a graph showing motion acceleration by time according to the present invention. FIG. 13 is an exemplary view showing a stand-to-sit motion according to the present invention, which corresponds to an area A of FIG. 12. FIG. 14 is an exemplary view showing a sit-to-stand motion according to the present invention, which corresponds to an area B of FIG. 12. When the chair stand test is carried out, the process of notifying test start and test explanation is the same as in the above-mentioned balance test, and therefore, an explanation of the process will be avoided.

Referring to FIG. 11, the chair stand test is carried out by repeating the sit-to-stand motion five times, without the use of the patient's hands (that is, with his or her arms folded on the chest), measuring the time taken at the last point, and applying scores according to the measured time. For example, if the time taken is greater than 60 seconds or the motion is not carried out, a zero point is applied, if the time taken is greater than 16.7 seconds, one point, if the time taken is in the range between 13.7 and 16.69 seconds, two points, if the time taken is in the range between 11.2 and 13.69 seconds, three points, and if the time is less than 11.19 seconds, four points.

Further, the chair stand test is carried out by using the up and down acceleration az or the total acceleration signal magnitude as analysis values to thus sense the minimum peak value and the maximum peak value of the analysis values, so that the motions of the patient can be recognized. Referring to FIG. 12, while the sit-to-stand motion is carried out by the patient five times, the time taken is measured at the last point ⊗ after the motion has been repeated five times, and it can be appreciated that the time taken is for example 40 seconds. Further, the stand-to-sit motion and the sit-to-stand motion are repeatedly carried out, while having one pair of a minimum peak value (hereinafter, referred to as min value) and a maximum peak value (hereinafter, referred to as max value) in the total acceleration signals. In this case, the stand-to-sit motion and the sit-to-stand motion are performed for about 2 to 3 seconds, respectively.

In specific, if the min value occurs earlier than the max value so that the difference between the two values is greater than a preset threshold value, the stand parameter detection module 125 determines (senses) that the patient performs the stand-to-sit motion. Further, the stand parameter detection module 125 sensing the stand-to-sit motion detects stand-to-sit parameters from the motion acceleration signals. For example, the detectable stand-to-sit parameters are listed in Table 6. Referring further to FIG. 13, at the time point where the first stand-to-sit motion is performed (the area A of FIG. 13 in the range of about 3.5 to 5.5 seconds), the time (a) taken to perform the stand-to-sit motion is about 2 seconds, and the min value (c) is sensed earlier than the max value (b). In this case, a difference between the min value (c) and the max value (b) is greater than the preset threshold value, and accordingly, the motion is sensed as the stand-to-sit motion. The threshold value in FIG. 12 is arbitrarily set, and in some cases, therefore, it may be changed. Referring to FIG. 12, further, the value from the start point to the min value (c) is defined as the time (e) taken from the start to a trough value (d), and the value from the start point to the max value (b) is defined as the trough value (d).

TABLE 6

| | Parameter | Description |
|---|---|---|
| (a) | Time stand-to-sit | Time taken |
| (b) | Peak stand-to-sit | Peak value |
| (c) | Time peak stand-to- | Time taken from start to |
| | sit | peak value |
| (d) | Trough stand-to-sit | Trough value |
| (e) | Time trough stand- | Time taken from start to |
| | to-sit | trough value |
| (f) | ax low frequency | Low frequency power (0.5 to 3 |
| | power | Hz) |
| (g) | ax high frequency | High frequency power (3 to 8 |
| | power | Hz) |
| (h) | ay low frequency | Low frequency power (0.5 to 3 |
| | power | Hz) |
| (i) | ay high frequency | High frequency power (3 to 8 |
| | power | Hz) |
| (j) | az low frequency | Low frequency power (0.5 to 3 |
| | power | Hz) |
| (k) | az high frequency | High frequency power (3 to 8 |
| | power | Hz) |
| (l) | svm low frequency | Low frequency power (0.5 to 3 |
| | power | Hz) |
| (m) | svm high frequency | High frequency power (3 to 8 |
| | power | Hz) |

Further, if the max value occurs earlier than the min value so that the difference between the two values is greater than a threshold value, the stand parameter detection module 125 determines (senses) that the patient performs the sit-to-stand motion. Further, the stand parameter detection module 125, which has sensed the sit-to-stand motion, detects sit-to-stand parameters from the motion acceleration signals. For example, the detectable sit-to-stand parameters are listed in Table 7. Referring further to FIG. 14, at the time point where the last stand-to-sit motion is performed (the area B of FIG. 14 in the range of about 42 to 44 seconds), the time (a) taken to perform the sit-to-stand motion is about 2 seconds, and the max value (b) is sensed earlier than the min value (c). In this case, a difference between the max value (b) and the min value (c) is greater than a preset threshold value, the motion is sensed as the sit-to-stand motion. Referring to FIG. 14, further, the value from the start point to the max value (b) is defined as the trough value (d), and the value from the start point to the min value (c) is defined as the time (e) taken from the start point to the trough value (d).

TABLE 7

| | Parameter | Description |
|---|---|---|
| (a) | Time stand-to-sit | Time taken |
| (b) | Peak_ stand-to- | Peak value |
| | sit | |
| (c) | Time peak_ stand- | Time taken from start point |
| | to-sit | to peak value |
| (d) | Trough_stand-to- | Trough value |
| | sit | |
| (e) | Time | Time taken from start point |
| | trough_stand-to- | to trough value |
| | sit | |
| (f) | ax low frequency | Low frequency power (0.5 to |
| | power | 3 Hz) |
| (g) | ax high frequency | High frequency power (3 to 8 |
| | power | Hz) |
| (h) | ay low frequency | Low frequency power (0.5 to |
| | power | 3 Hz) |
| (i) | ay high frequency | High frequency power (3 to 8 |
| | power | Hz) |
| (j) | az low frequency | Low frequency power (0.5 to |
| | power | 3 Hz) |
| (k) | az high frequency | High frequency power (3 to 8 |
| | power | Hz) |
| (l) | svm low frequency | Low frequency power (0.5 to |
| | power | 3 Hz) |
| (m) | svmhigh | High frequency power (3 to 8 |
| | frequency power | Hz) |

So as to determine the frailty of the patient, generally, the conventional physical performance assessment device uses the SPPB that measures the patient's gait speed, chair stand, and balance to determine his or her physical performance. However, professionally trained personnel (hereinafter, referred to as medical staff) is needed in executing comprehensive geriatric assessment for older patients, and further, a lot of time is disadvantageously required for the assessment. Besides, the patient's physical performance is measured with the naked eye of the medical staff, and accordingly, the degree of accuracy in the assessment may be diminished. Further, the assessment results may be varied according to the patient's mood or condition upon the test, and the frailty of the patient is assessed through indirect data, thereby making the degree of accuracy in the assessment results deteriorated.

Further, the conventional physical performance assessment device is configured to attach a sensor to the lower limb of the body to collect the ground reaction force or to use two different force plates to collect ground reaction forces separated from each other. Accordingly, since the ground reaction forces for the left and right legs are collected, they have to be added to perform the patient's gait analysis, thereby disadvantageously making the work efficiency lowered. Besides, if the sensor is attached to the lower limb, the degree of accuracy in the test may become lower than that in the case where the sensor is attached to the upper body of the patient, thereby disadvantageously making it difficult to perform data analysis. Moreover, in the case where the ground reaction forces are collected through the force plates, poor portability may occur.

To the contrary, the physical performance assessment device according to the present invention is configured to allow only the motion acceleration sensor to be attached to the head, so that the patient's motions, which have been measured with the naked eye in the conventional practice, can be accurately analyzed. As shown in FIGS. 1a and 1b, the physical performance assessment device 100 attached to the patient's head can obtain the gait time points of the right and left legs and detect the gait parameters, so that the motions can be divided to thus perform the physical performance assessment with a minimum error rate.

According to the present invention, further, the patient's motion acceleration signals are collected and the gait motions are analyzed only through the motion acceleration sensor attached to the head (that is, only through the physical performance assessment device 100), thereby improving the assessment speed.

Moreover, the gait time points for the left and right legs can be distinguished from each other based on the motion acceleration collected from the motion acceleration sensor attached to the head, thereby increasing the conveniences of the assessment.

In addition, the physical performance assessment device 100 is configured to collect the up and down acceleration, the front and back acceleration, and/or the left and right acceleration so that the patient's motions can be completely analyzed to accurately detect his or her gait abnormality due to a disease. For example, the gait abnormality motion patterns of the patient having Parkinson's disease or alzheimer's disease can be detected, thereby previously preventing the accidents of the patient from happening. For example, the patients can be protected from fall accidents through the analysis of their gait abnormality motion patterns.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention. This application is intended to cover any variations, uses, or adaptations of the invention following the general principles thereof and including such departures from the present disclosure as come within known or customary practice in the art. It is intended that the specification and examples be considered as exemplary only, with a true scope and spirit of the invention being indicated by the following claims.

1. A physical performance assessment method using a physical performance assessment device having a motion measurement module for measuring a patient's motion acceleration signals through a motion acceleration sensor attached to the patient's head and a motion analysis module for analyzing the patient's physical performance based on the measured motion acceleration signals, the method comprising the steps of:
collecting the motion acceleration signals measured from the motion acceleration sensor attached to the patient's head through the motion measurement module and used to analyze the patient's motions; and
deriving a plurality of motion parameters based on the collected motion acceleration signals through the motion analysis module,
wherein the step of deriving the plurality of motion parameters comprises the steps of:
when a protocol for balance among the plurality of motion parameters is executed, collecting the patient's three-axis motion acceleration signals for side-by-side stance, tandem stance, and semi-tandem stance and detecting balance parameters capable of assessing the patient's balance performance from total acceleration signals where the three-axis motion acceleration signals are added;
when a protocol for gait speed among the plurality of motion parameters is executed, detecting single stance and double stance from the motion acceleration signals to detect gait parameters from the detected results; and
when a protocol for chair stand among the plurality of motion parameters is executed, detecting a minimum peak value and a maximum peak value from the up and down acceleration and the total motion acceleration signals to detect stand parameters through the relation between the minimum peak value and the maximum peak value.

2. The physical performance assessment method according to claim 1, further comprising the step of determining, when the protocol for the balance is executed, whether a balance test is completed based on the magnitude of the total acceleration signals with respect to a preset threshold value.

3. The physical performance assessment method according to claim 2, wherein when the magnitude of the total acceleration signals is greater than the preset threshold value, it is determined that the test fails or the patient quits in the middle of the test, the time taken is recorded up to the time point where the magnitude of the total acceleration signals is greater than the threshold value, and scores are applied to the recorded time according to preset score ranges.

4. The physical performance assessment method according to claim 3, wherein the magnitude of the total acceleration signals is a difference between a maximum peak value and a minimum peak value in the total acceleration signals.

5. The physical performance assessment method according to claim 1, further comprising the steps of:
detecting the patient's gait time points based on the gait parameters;
sensing a gait start point and a gait finish point based on the gait time points; and
assessing the patient's gait speed based on the time taken from the gait start point and the gait finish point.

6. The physical performance assessment method according to claim 1, wherein while the protocol for the chair stand is executed, when the minimum peak value is sensed earlier than the maximum peak value in the up and down acceleration and the total motion acceleration signals so that a difference between the minimum peak value and the maximum peak value is greater than a preset threshold value, it is determined that a stand-to-sit motion is performed.

7. The physical performance assessment method according to claim 1, wherein while the protocol for the chair stand is executed, when the maximum peak value is sensed earlier than the minimum peak value in the up and down acceleration and the total motion acceleration signals so that a difference between the minimum peak value and the maximum peak value is greater than the preset threshold value, it is determined that a sit-to-stand motion is performed.

8. The physical performance assessment method according to claim 6, wherein the stand parameters comprise stand-to-sit parameters for the stand-to-sit motion and sit-to-stand parameters for the sit-to-stand motion.

9. The physical performance assessment method according to claim 1, wherein the gait parameters comprise vertical ground reaction forces and peak and trough values of the vertical ground reaction forces.

10. The physical performance assessment method according to claim 1, wherein the gait parameters further comprise low frequency power in low frequency band and high frequency power in high frequency band, and according to a rate of the low frequency power with respect to the high frequency power, the patient's gait motion is determined.

11. The physical performance assessment method according to claim 10, wherein the rate of the low frequency power with respect to the high frequency power is calculated as a constant value by the following Mathematical expression 1,
[High-band frequency (square of total power in the 3-8 Hz band)]/[Low-band frequency (square of total power in the 0.5-3 Hz band)]=constant, and <Mathematical expression 1>
when the constant value is less than a preset constant value capable of determining the patient's gait motion, it is determined as a normal state, and when the constant value is greater than the preset constant value, it is determined that the patient's physical performance stability becomes deteriorated.

12. A physical performance assessment device comprising:
a motion measurement module for collecting a patient's motion acceleration signals for the patient's motion analysis through a motion acceleration sensor attached to the patient's head; and
a motion analysis module for deriving a plurality of motion parameters based on the collected motion acceleration signals,
wherein the motion analysis module collects, when a protocol for balance among the plurality of motion parameters is executed, the patient's three-axis motion acceleration signals for side-by-side stance, tandem stance, and semi-tandem stance to detect balance parameters capable of assessing the patient's balance performance from total acceleration signals where the three-axis motion acceleration signals are added, detects, when a protocol for gait speed among the plurality of motion parameters is executed, single stance and double stance from the motion acceleration signals to detect gait parameters from the detected results, and detects, when a protocol for chair stand among the plurality of motion parameters is executed, a minimum peak value and a maximum peak value from the up and down acceleration and the total motion acceleration signals to detect stand parameters through the relation between the minimum peak value and the maximum peak value.

13. The physical performance assessment device according to claim 12, wherein when the magnitude of the total acceleration signals is greater than the preset threshold value, it is determined that the test fails or the patient quits in the middle of the test, the time taken is recorded up to the time point where the magnitude of the total acceleration signals is greater than the threshold value, and scores are applied to the recorded time according to preset score ranges.

14. The physical performance assessment device according to claim 12, wherein the magnitude of the total acceleration signals is a difference between a maximum peak value and a minimum peak value in the total acceleration signals.

15. The physical performance assessment device according to claim 12, wherein when the minimum peak value is sensed earlier than the maximum peak value in the up and down acceleration and the total motion acceleration signals so that a difference between the minimum peak value and the maximum peak value is greater than the preset threshold value, it is determined that a stand-to-sit motion is performed.

16. The physical performance assessment device according to claim 12, wherein when the maximum peak value is sensed earlier than the minimum peak value in the up and down acceleration and the total motion acceleration signals so that a difference between the minimum peak value and the maximum peak value is greater than the preset threshold value, it is determined that a sit-to-stand is performed.

17. The physical performance assessment device according to claim 12, further comprising a notification module for providing test explanations to the form recognized by the patient such as sound, video, or the like so that through the test explanations, the patient executes the protocol.

18. The physical performance assessment method according to claim 7, wherein the stand parameters comprise stand-to-sit parameters for the stand-to-sit motion and sit-to-stand parameters for the sit-to-stand motion.
